(19) United States
(12) Patent Application Publication
(10) Pub. No.: US 2008/0044928 A1
(43) Pub. Date: Feb. 21, 2008

(54) Method for the Preparation of Ready-to-Use Support for Rapid Enzyme-Linked Immunosorbent Assay (Elisa)

(76) Inventors: Bharat Raghunath Char, Jalna (IN); Pankaj Rameschandra Bihani, Jalna (IN)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(21) Appl. No.: 10/583,751

(22) PCT Filed: Dec. 22, 2004
(86) PCT No.: PCT/IN04/00394

§ 371 Date: Apr. 3, 2007

(30) Foreign Application Priority Data
Dec. 23, 2003 (IN) 1600/DEL/2003

(51) Int. Cl. G01N 33/543

(52) U.S. Cl. 436/524

(57) ABSTRACT

The present invention provides a method for the preparation of ready-to-use solid support for ELISA for rapid identification and quantitative estimation of protein/antigen in the test samples, and performances of the assay itself. The invention also provides for a quick, accurate and stable estimation of protein/antigen in the test samples. The invention also provides an ELISA kit comprising of ready-to-use solid support along with wash buffers, chemical substrate, substrate buffer, stock solution, and positive and negative control samples.

### TECHNICAL FIELD

The present invention relates to a method for the preparation of ready-to-use solid support for ELISA for rapid identification and quantitative estimation of protein/antigen in the test samples, and performance of the assay itself. The invention also provides for a quick, accurate and stable estimation of protein/antigen in the test samples. The invention also provides a rapid ELISA kit comprising of ready-to-use solid support along with wash buffers, chemical substrate, substrate buffer, stock solution, and positive and negative control samples.

### BACKGROUND AND PRIOR ART REFERENCES

ELISA is a widely used method for the detection of specific proteins in a tissue sample. It involves the immobilization of an antibody (primary antibody) to a surface of substrate such as plastic, and detecting a specific antigen (protein) via binding to the immobilized antibody, followed by addition of secondary antibody or antibodies, the latter being conjugated to enzymes such as alkaline phosphatase or horseradish peroxidase. Addition of a chemical substrate of the enzyme results in the development of a coloured reaction product, which indicates the presence of the antigen of interest in the sample.

ELISA is a time-tested and robust method, and is used for the detection of a multitude of proteins from a large number of sources. Commercial suppliers of ELISA products provide plates coated with primary antibody. The user has to then follow a procedure containing a series of steps involving addition of the sample, addition of secondary antibody or antibodies in sequence, several buffer wash steps in between each antibody addition step, and finally the detection step via substrate addition. The established procedures are often time-consuming and necessitate the formulation of various buffers and solutions. It often takes up to 24 hours to complete the protocol and obtain results.

There are a number of variations in ELISA and these determine the number of steps involved or time taken to complete the assay. For example, the secondary antibody may be conjugated to alkaline phosphatase or horseradish peroxidase, in which case the substrate for colour development can be added immediately after the secondary antibody. This is known as direct ELISA. However, if the secondary antibody is unconjugated, then a third conjugated antibody is needed for colour detection. This type of assay is known as indirect ELISA. Antibodies (conjugated or otherwise) are either commercially available from vendors or need to be custom-produced. Thus one of the drawbacks in the established ELISA technique is to procure and maintain a stock of the necessary antibodies.

A search of the patent literature revealed one patent, WO02090983, Quantitative One-Step Immunoassay in Lyophilised Form, Inventors: Rech-Weichselbraun, I. (AT) and Staude M. (AT) directly relevant to the present invention. In this patent, proteins, antibodies and reaction enhancing agents such as biotin and streptavidin are immobilised on the plate along with coating antibody. A rehydration step is followed by sample addition and detection steps. This eliminates a number of intermediate steps. However, there are some significant differences from the present invention. These are: 1) The applications of the method described in WO02090983 are limited to detection of cytokines and related molecules used in cancer research, whereas the present invention relates to detection of proteins in plant tissues. 2) Assay times in WO02090983 vary significantly for each application, and can be as high as 250 min, whereas in the present invention assay times do not exceed 150 min. 3) WO02090983 uses a biotin-streptavidin system to enhance the sensitivity of the assay, whereas the present invention does not require this additional set of reagents.

Another related patent found was WO0214868, A rapid method for microwave mediated enzyme-linked immunosorbent assays, Publication date: Feb. 21, 2002, Inventor(s): Sharma Gainda Lal (In); Nahar Pradeep (In); Bora Utpal (In); involving the use of a microwave oven to enhance the ELISA. However the key requirement of the microwave oven increases costs and necessitates the optimisation of protocols for each protein of interest, as each antigen to be utilised in such a method may have a different tolerance to heating by microwave radiation. Heat labile proteins would suffer adverse effects upon microwave treatment necessitating a modification in the protocol.

### OBJECTS OF THE INVENTION

The main object of the present invention is to provide a method for preparing a ready-to use solid support for rapid ELISA.

Another object of the present invention is to provide a ready-to-use solid support for rapid quantification of protein/antigen in test samples.

Another object of the present invention is to provide for a quick, accurate and stable estimation of protein/antigen in the test samples.

Another object of the method is to demonstrate the rapid performance of the method.

Still another object of the present invention is to provide an ELISA kit containing ready-to-use solid support for rapid identification of protein/antigen in the test sample.

Yet another object of the invention is to provide an ELISA kit containing ready-to-use solid support for rapid quantitative estimation of protein/antigen in the test sample.

Another object of the invention is to reduce the number of steps in the procedure that an end-user has to perform in an ordinary ELISA.

### SUMMARY OF THE INVENTION

In accordance to the objectives, the present invention provides a method for the preparation of ready-to-use solid support for ELISA for rapid identification and quantitative estimation of protein/antigen in the test samples and performance of the assay itself. The invention also provides for a quick, accurate and stable estimation of protein/antigen in the test samples. The invention also provides an ELISA kit comprising of ready-to-use solid support along with wash buffers, chemical substrate, substrate buffer, stock solution, and positive and negative control samples.

### DETAILED DESCRIPTION OF THE INVENTION

Accordingly, the present invention provides a method for preparing ready-to-use solid support for rapid ELISA, wherein the said method comprises steps of:

a) adding a first monoclonal antibody dissolved in coating buffer to the wells of the solid support, incubating the solid support at about 35 to 40° C. for a period ranging between about 12 and 14 hours for binding to the solid support;

b) washing the solid support of step (a), with a washing buffer to remove the unbound monoclonal antibody;

c) adding a stabilizer solution to the wells of the solid support of step (b), incubating for a period ranging between 12 and 14 hours at about 35 to 40° C.;

d) decanting to remove the stabilizer solution of step (c), and completely drying the wells of the solid support;

e) adding to the wells of the solid support of step (d), an appropriate second antibody and an appropriate third antibody conjugated to an enzyme dissolved in a suitable buffer containing the blocking agent; and

f) freeze drying the plate of step (e), storing the plate in a sealed pack at a temperature range of about 4-8° C. for ready-to-use.

One embodiment of the present invention is a ready-to-use solid support consisting of a bound antibody, wherein said antibody is capable of forming a first antigen-antibody complex with sample antigen/protein, a second antibody forming an antigen-antibody complex with the said sample antigen/protein and a detection antibody having a label which selectively binds to the second antibody.

The first monoclonal antibody is raised against the protein/antigen to be detected and the second antibody used is polyclonal antibody IgG raised against protein/antigen to be detected.

The third antibody is selected from the group consisting of polyclonal whole IgG conjugated to an enzyme, wherein whole IgG may be obtained from class Mammalia or class Aves.

The first monoclonal antibody used is selected from a group consisting of monoclonal antibodies raised against Cry proteins and monoclonal antibodies against 5-enolpyruvylshikimate-3-phosphate synthase, wherein Cry protein is preferably selected from Cry1Ab, Cry1Ac Cry2Ab, Cry 9A, Cry 9B and Cry 9C.

Another embodiment of the present invention is that the coating buffer used is selected from the group consisting of carbonate buffer and phosphate buffer, having pH in the range of 9.0-9.8.

Another embodiment of the invention is that the first monoclonal antibody used is selected from the group consisting Cry proteins such as of but not limited to Cry1Ab, Cry1Ac Cry2Ab, Cry 9A, Cry 9B and Cry 9C and 5-enolpyruvylshikimate-3-phosphate synthase (EPSPS).

Another embodiment of the invention is that the washing buffer used is phosphate-buffered saline having a pH in the range of 6.8-7.2.

Another embodiment of the invention is that the stabilizer used is selected from a group consisting of a Phosphate-Buffered Saline, Fish Gelatin and Glycerol mixture and a Tris-buffer, Fish Gelatin and Glycerol mixture.

Another embodiment of the invention is that it provides a method, wherein the blocking agent used is selected from the group consisting of ovalbumin, bovine serum albumin, bovine nonfat milk powder, casein, fish gelatin, porcine gelatin and lambda-carrageenan.

Another embodiment of the invention is that the solid support used is selected from the group consisting of ELISA plate and microwell plate.

Another embodiment of the invention is that the material for the solid support used is either polystyrene or polypropylene.

Another embodiment of the invention is that the solid support used is polystyrene.

Another embodiment of the invention is that the second antibody is selected from the group consisting of goat polyclonal IgG raised against Cry1Ac, goat polyclonal IgG raised against Cry2Ab and goat polyclonal IgG raised against 5-enolpyruvylshikimate-3-phosphate synthase (EPSPS).

Another embodiment of the invention is that the third antibody is selected from the group consisting of polyclonal whole IgG conjugated to an enzyme. The source of this polyclonal whole IgG can be Class Mammalia or Class Aves.

Another embodiment of the invention is that the enzyme used is selected from the group consisting of alkaline phosphatase and horseradish peroxidase.

Another embodiment of the present invention is that it provides a rapid method for performing ELISA using ready-to-use solid support, the said method comprising steps of:
a) reconstituting the ready to use plates by adding appropriate amount of distilled water;
b) adding to the plate of step (a), samples containing antigen/protein to be tested dissolved in a suitable buffer, incubating the plate at about 37° C. for about one hour for forming an immunocomplex with the bound first antibody;
c) washing the plate of step (b) with a suitable washing buffer to remove the unbound antigen;
d) adding to the plate of step (c), a buffer containing chemical substrate and incubating for about 30 minutes in dark at room temperature; and
e) detecting for the presence of the antigen by measuring absorbance in step (d) at a suitable wavelength

Another embodiment of the present invention is that the wavelength suitable for measuring the absorbance is in the range of 400-700 nm.

Another embodiment of the present invention is that it provides a method, wherein the chemical substrate is selected from the group consisting of para-nitrophenol phosphate (pNPP), Nitro Blue Tetrazolium/5-Bromo-4-Chloro-3-Indolyl Phosphate (NBT/BCIP), 2,2′-Azino-bis(3-Ethylbenz-thiazoline-6-Sulfonic Acid) (ABTS), o-Pheenylenediamine (OPD), 3,3′-5,5′-Tetramethylbenzidine (TMB), o-Dianisidine, and 5-Aminosalicylic Acid (5AS).

One embodiment of the present invention is that it provides a rapid ELISA kit comprising of
a) a ready-to-use solid support for detection of protein or antigen to be tested,
b) wash buffers,
c) chemical substrate,
d) substrate buffer,
e) stop solution,
f) positive and negative control samples, and
g) an instruction manual

Another embodiment of the invention provides a ready-to-use solid support for detection of protein or antigen

Yet another embodiment of the present invention is that it provides a quick, accurate and stable estimation of protein/antigen in the test samples.

### Novelty and Inventive Step

The key inventive steps and problems overcome are:
1. A novel method by which all antibodies required for the detection are made available to the assay in the wells of the ELISA plate. In order for the assay to work, a series of steps involving freeze-drying and addition of protein stabilizers in a precise, sequential manner has been devised, allowing the immobilisation or impregnation of the antibodies onto the ELISA plate.
2. Leading from the above, another key inventive step overcoming earlier problems is that of loss of viability of impregnated proteins. This occurs due to surface denaturation of the immobilised proteins possibly by means of oxidation or water loss [ref. Ansari A A, Hattilkudur N S, Joshi S R, Medeira M A. ELISA solid phase: stability and binding characteristics. J Immunol Methods. 84:117-24(1985)]. The present inventive steps prevent the surface denaturation of proteins immobilised to the solid support, and the procedures described herein enable the antibodies to be viable for use in the ELISA. This has been achieved by a series of coating and drying steps, alternating an antibody layer with a layer of stabiliser, and then freeze-drying or drying the material to retain its activity over a period of time.
3. A reconstitution step is incorporated in the ELISA protocol to allow the antibodies to be accessible to the protein of interest in the sample. This step is necessary to enable the immobilised, stabilised proteins to become optimally functional for the ELISA to work. No prior art was found which mentioned the stabilising of multiple proteins, i.e. “layering”, on a solid support for storage and then use in an ELISA. This invention describes a novel way in which more than one protein can be successfully applied to the solid support, and subsequently used after a substantial period of time by means of a simple reconstitution step. For the end-user, this overcomes the problem of having to obtain conjugated or unconjugated antibodies from various sources, thereby overcoming one of the main drawbacks in the conventional method.
4. The present invention obviates the need for additional equipment such as a microwave oven as described in WO0214868, A rapid method for microwave mediated enzyme-linked immunosorbent assays, Publication date: Feb. 21, 2002, Inventor(s): Sharma Gainda Lal (In); Nahar Pradeep (In); Bora Utpal (In). Secondly, it also obviates the need to use biotin-streptavidin linked antibodies as described in WO02090983, Quantitative One-Step Immunoassay in Lyophilised Form, Inventors: Rech-Weichselbraun, I. (AT) and Staude M. (AT).
5. The invention enables a reduction in the number of steps that an end-user has to perform in an ELISA. All reagents needed to perform the assay are impregnated on to the plate by the use of particular stabilizing processes as well as particular protein stabilizers. The user only performs sample addition, wash and detection steps. The user adds a reconstitution buffer to the wells of the ELISA plate, followed by the samples to be tested. After an incubation period, the samples are discarded, and the plates washed. A chemical substrate is then added, which results in the appearance of a coloured reaction product in positive samples and a lack of colour in negative samples.
6. The present invention enables the user to perform a rapid ELISA in one step, as only samples need to be added to the ready-to-use plate prior to the detection step.
7. The present invention also provides for a quick, accurate and stable estimation of protein/antigen in the test samples.

The following examples are for understanding the invention and should not be construed to limit the scope of the invention.

### EXAMPLES

### Example 1 (Total Time for Assay: 150 Min)

This method can be used for the detection of protein Cry1Ac in cottonseed and cotton leaf extracts in a qualitative manner, as indicated in the protocol below.

### Steps Involved:

1) Preparation of buffers

2) ELISA plate coating with Cry1Ac mAb

3) Addition of Ab2 & Ab3

4) Sample preparation

5) Assay

### 1) Preparation of Buffers:

a) Carbonate Buffer:

| | |
|---|---|
| Sodium carbonate | 1.59 g |
| Sodium bicarbonate | 2.93 g |
| Sodium chloride | 8.77 g |
| D/w | 1 L |

After preparing store at 4° C. (cold room)

b) 10× PBST: (pH 7.4)

| | |
|---|---|
| Sodium chloride | 80.0 g |
| Sodium phosphate dibasic | 11.50 g |
| Potassium chloride | 2.0 g |
| Potassium dihydrogen phosphate | 2.0 g |
| D/w | 1 L |

Add 5 ml Tween 20 to 1 L volume.

c) 1× PBST:
Take 100 ml of 10× PBST dilute it to 1 L by adding D/w.

d) 10× PBSTO:
Add 0.5 gm ovalbumin in 10 ml 10× PBST.
Store the solution in 4° C. refrigerator.

e) Substrate Buffer:
Prepare 5% diethanolamine (DEA) in Milli Q, adjust the pH with concentrated HCl for 1 hr till the required pH is attained.

f) Substrate: (Mg/Ml Conc.)
Take 25 ml substrate buffer; add 25 mg pNPP to it. Mix well.

### Note: Substrate should be prepared freshly.

Remove pNPP bottle at least 20 min. before use from 4° C. After preparing substrate buffer with substrate keep it in dark for 10 min. before use.

g) Stabilizer:

| | |
|---|---|
| 10X PGFG/TGFG | 2.5 ml |
| 10X PBS | 2.5 ml |
| D/w | 20 ml |

### 2) Coating ELISA Plates with Cry1Ac Monoclonal Antibodies:

Add 250 μl of Cry1Ac monoclonal antibody per well of the Elisa plate at a concentration of 2 μg/ml.

### Procedure:

Mix 12.8 μl mAb in 25 ml carbonate buffer. Using multichannel pipetter, add 250 μl in each well of the plate. Incubate the plate O/N at 4° C. Give two quick washes with 1× PBST. Pat dry on blotting paper. Add stabilizer, 250 μl/well, and incubate O/N at 4° C. Decant the plate & allow it to air dry completely.

### 3) Addition of Ab2 & Ab3:

Concentration of Ab2: 1:10,000

Concentration of Ab2: 1:5000

### Procedure:

Pipette out 1.5 μl of Ab2 & 3.8 μl of Ab3 stock in an eppendorf tube containing 1.5 ml of 10× PBSTO. Mix well and add 15 μl/well using a multichannel pipetter. Freeze-dry the plate for 15 min. Store the freeze-dried plates in sealed pack containing desiccant at 4° C., till further use.

### 4) Sample Preparation

### Note: Avoid cross-contamination between samples

For seed extracts: Imbibe cotton seeds overnight in water. Remove seed coat and cut each seed to be tested in half with a clean blade. Place one half of the seed in a microcentrifuge tube and add 500 μl 1× PBST. Crush with a pestle for 30 seconds. Spin for 30 sec in a microcentrifuge, and use 100 μl of each extract per well, taking care to avoid the pellet.

For leaf extracts: Punch out 2 leaf discs with a mcf tube by placing a leaf between the lid and the tube opening and closing the lid onto the leaf. Add 500 μl× PBST. Crush with a pestle for 30 seconds. Allow to stand for few minutes, and use 100 μl of each extract per well, taking care to avoid the pellet.

### 5) Assay:

Reconstitute the freeze-dried plate for 30 min. by adding 150 μl/well Milli Q water. After reconstitution, add samples, 100 μl/well. Incubate the plate at 37° C. for 1 hr. Give four quick washes with 1× PBST. Pat dry. Add substrate, 250 μl/well, & incubate it for 30 min. dark at room temperature. Read the absorbance on an ELISA reader at 405 nm.

### Sample Plate Result for Cry1Ac:

### Results:

The grid below represents a 96-well ELISA plate in which Cry1Ac expressing cotton leaf samples have been tested using the inventive method. “Blank” refers to wells in which no cotton leaf extract has been added. This gives the baseline absorbance reading for the experiment and is subtracted from all sample readings. Absorbance values provided have blank values already subtracted (hence the blank wells read 0.0). “+ve” refers to known Cry1Ac expressing samples. Unmarked wells are equivalent to Blank wells, i.e.; no cotton leaf extract was added. A reading of above 0.2 is considered a positive reading. Plates prepared by the present inventive method were used in an experiment to determine whether known positive and negative samples could be accurately detected. In this example 100% (28/28) of the samples were detected accurately.

### Samples Added:

| | 1 | 2 | 3 | 4 | 5 | 6 | 7 | 8 | 9 | 10 | 11 | 12 |
|---|---|---|---|---|---|---|---|---|---|---|---|---|
| A | Blank | +ve | | +ve | | | | +ve | | +ve | | |
| B | +ve | | +ve | | | +ve | +ve | | +ve | | | +ve |
| C | −ve | | | | | | −ve | | | | | |
| D | | +ve | +ve | +ve | +ve | +ve | | +ve | +ve | +ve | +ve | +ve |
| E | | | | | | | | | | | | |
| F | | −ve | | −ve | | −ve | | −ve | | −ve | | −ve |
| G | | | | | | | | | | | | |
| H | | | | | | | | | | | | |

### Absorbance Values:

The plate represented above gave absorbance values as below

| | 1 | 2 | 3 | 4 | 5 | 6 | 7 | 8 | 9 | 10 | 11 | 12 |
|---|---|---|---|---|---|---|---|---|---|---|---|---|
| A | 0.000 | 0.890 | | 1.220 | | | | 0.836 | | 1.082 | | |
| B | 0.935 | | 0.882 | | | 0.704 | 0.802 | | 1.025 | | | 1.007 |
| C | 0.014 | | | | | | 0.006 | | | | | |
| D | | 0.538 | 0.652 | 0.417 | 0.482 | 0.545 | | 0.666 | 0.668 | 0.612 | 0.529 | 0.907 |
| E | | | | | | | | | | | | |
| F | | 0.014 | | −0.001 | | 0.024 | | 0.013 | | −0.109 | | 0.131 |
| G | | | | | | | | | | | | |
| H | | | | | | | | | | | | |

### Example 2 (Total Time: 150 min)

This method can be used for the detection of protein Cry2Ab in cottonseed and cotton leaf extracts in a qualitative manner, as indicated in the protocol below.

### Steps Involved:

1) Preparation of buffers

2) ELISA plate coating with Cry2Ab mAb

3) Addition of Ab2 & Ab3

4) Sample preparation

5) Assay

### 1) Preparation of Buffers:

Please refer to Example 1

### 2) Coating ELISA Plates with Cry2Ab Monoclonal Antibodies:

Add 250 μl of Cry2Ab monoclonal antibody per well of the Elisa plate at a concentration of 2 μg/ml.

### Procedure:

Mix 13.3 μl mAb in 25 ml carbonate buffer. Using multichannel pipetter, add 250 μl in each well of the plate. Incubate the plate O/N at 4° C. Give two quick washes with 1× PBST. Pat dry on blotting paper. Add stabilizer, 250 μL/well, and incubate O/N at 4° C. Decant the plate & allow it to air dry completely.

### 3) Addition of Ab2 & Ab3:

Concentration of Ab2: 1:4000

Concentration of Ab2: 1:5000

### Procedure:

Pipette out 1.5 μl of Ab2 & 3.8 μl of Ab3 stock in an eppendorf tube containing 2 ml of 10× PBSTO.

Mix well and add 15 μl/well using a multichannel pipetter. Freeze-dry the plate for 15 min. Store the freeze-dried plates in sealed pack containing desiccant at 4° C., till further use.

### 4) Sample Preparation

Note: Avoid cross-contamination between samples

For seed extracts: Imbibe cotton seeds overnight in water. Remove seed coat and cut each seed to be tested in half with a clean blade. Place one half of the seed in a microcentrifuge tube and add 500 μl 1× PBST. Crush with a pestle for 30 seconds. Spin for 30 sec in a microcentrifuge, and use 100 μl of each extract per well, taking care to avoid the pellet.

For leaf extracts: Punch out 2 leaf discs with a mcf tube by placing a leaf between the lid and the tube opening and closing the lid onto the leaf. Add 500 μl×PBST. Crush with a pestle for 30 seconds. Allow to stand for few minutes, and use 100 μl of each extract per well, taking care to avoid the pellet.

### 5) Assay:

Reconstitute the freeze-dried plate for 30 min. by adding 150 μl/well Milli Q. After reconstitution, add samples, 100 μl/well. Incubate the plate at 37° C. for 1 hr. Give four quick washes with 1× PBST. Pat dry. Add substrate, 250 μl/well, & incubate it for 30 min. dark at RT. Read the absorbance on ELISA reader at 405 nm.

### Sample Plate Result for Cry2Ab:

### Results:

The grid below represents a 96-well ELISA plate in which Cry2Ab expressing cotton leaf samples have been tested using the inventive method. “Blank” refers to wells in which no cotton leaf extract has been added. This gives the baseline absorbance reading for the experiment and is subtracted from all sample readings. Absorbance values provided have blank values already subtracted (hence the blank wells read 0.0). “+ve” refers to known Cry2Ab expressing samples. Unmarked wells are equivalent to Blank wells, i.e.; no cotton leaf extract was added. A reading of above 0.2 is considered a positive reading. Plates prepared by the present inventive method were used in an experiment to determine whether known positive and negative samples could be accurately detected. In this example 98.3% (59/60) of the samples were detected accurately.

| | 1 | 2 | 3 | 4 | 5 | 6 | 7 | 8 | 9 | 10 | 11 | 12 |
|---|---|---|---|---|---|---|---|---|---|---|---|---|
| A | Blank | +ve | −ve | +ve | | +ve | blank | +ve | −ve | +ve | | +ve |
| B | +ve | +ve | | +ve | −ve | +ve | +ve | +ve | | +ve | −ve | +ve |
| C | −ve | +ve | −ve | +ve | | +ve | −ve | +ve | −ve | +ve | | +ve |
| D | | +ve | | +ve | −ve | +ve | | +ve | | +ve | −ve | +ve |
| E | | +ve | −ve | +ve | | | | +ve | −ve | +ve | | |
| F | | +ve | | +ve | −ve | | | +ve | | +ve | −ve | |
| G | | +ve | −ve | +ve | | | | +ve | −ve | +ve | | |
| H | | +ve | | +ve | −ve | | | +ve | | +ve | −ve | |

### Absorbance Values:

The plate represented above gave absorbance values as below

| | 1 | 2 | 3 | 4 | 5 | 6 | 7 | 8 | 9 | 10 | 11 | 12 |
|---|---|---|---|---|---|---|---|---|---|---|---|---|
| A | 0.000 | 0.313 | −0.027 | 0.624 | | 0.668 | −0.017 | 0.271 | 0.016 | 0.573 | | 0.668 |
| B | 0.501 | 0.483 | | 0.296 | −0.030 | 0.679 | 0.417 | −0.027 | | 0.304 | −0.037 | 0.838 |
| C | 0.013 | 0.645 | −0.037 | 0.737 | | 0.685 | −0.043 | 0.544 | | 0.722 | | 0.809 |
| D | | 0.634 | | 0.633 | −0.037 | 0.452 | | 0.560 | −0.005 | 0.687 | −0.027 | 0.511 |
| E | | 0.809 | −0.043 | 0.723 | | | | 0.804 | | 0.806 | | |
| F | | 0.944 | | 0.824 | −0.044 | | | 0.785 | 0.012 | 0.849 | −0.031 | |
| G | | 0.631 | −0.038 | 0.851 | | | | 0.722 | | 1.244 | | |
| H | | 0.469 | | 0.577 | −0.019 | | | 0.487 | 0.021 | 0.574 | −0.032 | |

### Example 3 (Total Time: 150 Min)

This method can be used for the detection of protein EPSPS in cottonseed and cotton leaf extracts in a qualitative manner, as indicated in the protocol below.

### Steps Involved:

1) Preparation of buffers

2) ELISA plate coating with EPSPS mAb

3) Addition of Ab2 & Ab3

4) Sample preparation

5) Assay

### 1) Preparation of Buffers:

Please refer to Example 1

2) Coating ELISA Plates with EPSPS Monoclonal Antibodies:

Add 250 μl of EPSPS monoclonal antibody per well of the Elisa plate at a concentration of 2 μg/ml.

### Procedure:

Mix 13.3 μl mAb in 25 ml carbonate buffer. Using multichannel pipetter, add 250 μl in each well of the plate. Incubate the plate O/N at 4° C. Give two quick washes with 1× PBST. Pat dry on blotting paper. Add stabilizer, 250 μl/well, and incubate O/N at 4° C. Decant the plate & allow it to air dry completely.

### 3) Addition of Ab2 & Ab3:

Concentration of Ab2: 1:20,000

Concentration of Ab2: 1:8000

### Procedure:

Pipette out 1.0 μl of Ab2 & 3.1 μl of Ab3 stock in an eppendorf tube containing 2 ml of 10× PBSTO.

Mix well and add 15 μl/well using a multichannel pipetter. Freeze-dry the plate for 15 min. Store the freeze-dried plates in sealed pack containing desiccant at 4° C., till further use.

### 4) Sample Preparation

Note: Avoid cross-contamination between samples

For seed extracts: Imbibe cotton seeds overnight in water. Remove seed coat and cut each seed to be tested in half with a clean blade. Place one half of the seed in a microcentrifuge tube and add 500 μl 1× PBST. Crush with a pestle for 30 seconds. Spin for 30 sec in a microcentrifuge, and use 100 μl of each extract per well, taking care to avoid the pellet.

For leaf extracts: Punch out 2 leaf discs with a mcf tube by placing a leaf between the lid and the tube opening and closing the lid onto the leaf. Add 500 μl×PBST. Crush with a pestle for 30 seconds, allow to stand for few minutes, and use 100 μl of each extract per well, taking care to avoid the pellet.

Assay: Reconstitute the freeze-dried plate for 30 min by adding 150 μl/well Milli Q. After reconstitution, add samples, 50 μl/well. Incubate the plate at 37° C. for 1 hr. Give four quick washes with 1× PBST. Pat dry. Add substrate, 250 μl/well, & incubate it for 30 min. dark at RT. Read the absorbance on ELISA reader at 405 nm.

### Sample Plate Result for EPSPS:

### Results:

The grid below represents a 96-well ELISA plate in which EPSPS expressing cotton leaf samples have been tested using the inventive method. “Blank” refers to wells in which no cotton leaf extract has been added. This gives the baseline absorbance reading for the experiment and is subtracted from all sample readings. Absorbance values provided have blank values already subtracted (hence the blank wells read 0.0).“+ve” refers to known EPSPS expressing samples. Unmarked wells are equivalent to Blank wells, i.e.; no cotton leaf extract was added. A reading of above 0.1 is considered a positive reading. Plates prepared by the present inventive method were used in an experiment to determine whether known positive and negative samples could be accurately detected. In this example 100% ( 38/38) of the samples were detected accurately.

| | 1 | 2 | 3 | 4 | 5 | 6 | 7 | 8 | 9 | 10 | 11 | 12 |
|---|---|---|---|---|---|---|---|---|---|---|---|---|
| A | Blank | +ve | +ve | −ve | −ve | −ve | −ve | −ve | −ve | −ve | | |
| B | −ve | | −ve | | −ve | | −ve | | | | | |
| C | | +ve | +ve | −ve | −ve | −ve | −ve | −ve | −ve | −ve | | |
| D | | +ve | | +ve | | +ve | | +ve | | | | |
| E | | +ve | | +ve | | +ve | | +ve | | | | |
| F | | +ve | +ve | +ve | +ve | | | | | | | |
| G | | | | | | | | | | | | |
| H | | +ve | +ve | +ve | +ve | | | | | | | |

### Absorbance Values:

The plate represented above gave absorbance values as below

| | 1 | 2 | 3 | 4 | 5 | 6 | 7 | 8 | 9 | 10 | 11 | 12 |
|---|---|---|---|---|---|---|---|---|---|---|---|---|
| A | 0.000 | 0.364 | 0.113 | 0.057 | 0.021 | 0.021 | 0.007 | 0.009 | 0.004 | 0.007 | | |
| B | 0.019 | | 0.055 | | −0.004 | | −0.004 | | | | | |
| C | | 0.358 | 0.105 | 0.056 | 0.010 | 0.008 | −0.001 | −0.003 | −0.004 | −0.005 | | |
| D | | 0.817 | | 1.072 | | 1.268 | | 0.484 | | | | |
| E | | 0.827 | | 1.109 | | 1.240 | | 0.479 | | | | |
| F | | 1.031 | 0.446 | 0.228 | 0.105 | | | | | | | |
| G | | | | | | | | | | | | |
| H | | 1.119 | 0.552 | 0.243 | 0.109 | | | | | | | |

### ADVANTAGES OF THE PRESENT INVENTION

The present invention relates to a process in which ELISA plates are provided to the user in a form in which only sample addition, wash and detection steps are required.

The advantages are:
1. A number of steps are reduced such as sequential antibody addition, and buffer washes.
2. There is no need for the end-user to purchase any antibodies given that all reagents required for the detection are present on the plate, except the sample itself, and the substrate required for colour production.
3. The assay is equally sensitive as other, more time-consuming or cumbersome protocols.
4. The method provides for a quick, accurate and stable estimation of protein/antigen in the test samples.

### REFERENCES

WO02090983, Quantitative One-Step Immunoassay in Lyophilised Form, Publication date: Nov. 14, 2002, Inventors: Rech-Weichselbraun, I. (AT) and Staude M. (AT)
WO0214868, A rapid method for microwave mediated enzyme-linked immunosorbent assays, Publication date: Feb. 21, 2002, Inventors: Sharma, Gainda Lal (In); Nahar, Pradeep (In); Bora, Utpal (In)
Ansari A A, Hattikudur N S, Joshi S R, Medeira M A. ELISA solid phase: stability and binding-characteristics. J Immunol Methods. 84:117-24(1985)

1. A method for preparing ready-to-use solid support for rapid ELISA, wherein the said method comprises addition of first monoclonal antibody, washing with buffer to remove unbound monoclonal antibody adding a stabilizer, removing excess stabilizer, air-drying of the bound stabilizer, addition of an appropriate second antibody and enzyme linked conjugate as third antibody together dissolved in buffer, lyophilising the said protein mixture and storing in a sealed package at a specified temperature.

2. A method as claimed in claim 1, wherein the first monoclonal antibody is raised against the protein/antigen to be detected.

3. A method as claimed in claim 1, wherein the first monoclonal antibody used is selected from a group consisting of monoclonal antibodies raised against Cry proteins and monoclonal antibodies against 5-enolpyruvylshikimate-3-phosphate synthase, wherein Cry protein is preferably selected from Cry1Ab, Cry1Ac Cry2Ab, Cry 9A, Cry 9B and Cry 9C.

4. A method as claimed in claim 1, wherein the buffer used for washing is phosphate buffer saline having a pH in the range of 6.8-7.2.

5. A method as claimed in claim 1, wherein buffer used for dissolving second and third antibody is selected from a group consisting of carbonate buffer and phosphate buffer, having pH in the range of 9.0-9.8.

6. A method as claimed in claim 1, wherein the stabilizer used is selected from a group consisting of Phosphate Buffered Saline, Fish Gelatin and Glycerol mixture and a Tris-buffer, Fish Gelatin and Glycerol mixture.

7. A method as claimed in claim 1, wherein the drying method used is either freeze drying or lyophilization.

8. A method as claimed in claim 1, wherein the blocking agent used is selected from the group consisting of ovalbumin, bovine serum albumin, bovine nonfat milk powder, casein, fish gelatin, porcine gelatin and lambda-carrageenan.

9. A method as claimed in claim 1, wherein the solid support used is selected from the group consisting of ELISA plate and microwell plate.

10. A method as claimed in claim 1, wherein the material for the solid support used is either polystyrene or polypropylene.

11. A method as claimed in claim 9, wherein the solid support is made of polystyrene.

12. A method as claimed in claim 1, wherein second antibody used is polyclonal antibody IgG raised against protein/antigen to be detected.

13. A method as claimed in claim 1, wherein second antibody used is polyclonal antibody IgG raised against corresponding Cry protein or IgG raised against 5-enolpyruvylshikimate-3-phosphate synthase.

14. A method as claimed in claim 1, wherein third antibody used is selected from the group consisting of polyclonal whole IgG conjugated to an enzyme, wherein whole IgG may be obtained from class Mammalia or Aves.

15. A method as claimed in claim 14, wherein the enzyme used is selected from a group consisting of alkaline phosphatase and horseradish peroxidase.

16. A rapid method for performing ELISA using ready-to-use solid support of claim 1 said method comprising steps of reconstituting the ready to use plates by adding appropriate amount of distilled water, adding test samples containing antigen/protein are dissolved in a suitable buffer, washing the plate after incubating for a required time period, followed by washing with suitable buffer, adding to the plate required chemical substrate and detecting for the presence of the antigen by measuring absorbance at a suitable wavelength.

17. A method as claimed in claim 16, wherein the chemical substrate is selected from the group consisting of para-nitrophenol phosphate, Nitro Blue Tetrazolium-5-Bromo-4-Chloro-3-Indolyl Phosphate, 2,2′-Azino-bis (3-Ethylbenz-thiazoline-6-Sulfonic Acid), o-Phenylenediamine, 3,3′-5,5′-Tetramethylbenzidine, o-Dianisidine and 5-Aminosalicylic Acid.

18. An immunoassay kit comprising of ready to use solid support of claim 1 for rapid ELISA.

19. A ready-to-use solid support of claim 1 for detection of protein or antigen
